(19) United States
(12) Patent Application Publication
(10) Pub. No.: US 2026/0240521 A1
(43) Pub. Date: Aug. 20, 2026

(54) INTRAVASCULAR ULTRASOUND PROBE ASSEMBLIES AND RELATED SYSTEMS AND METHODS

(71) Applicant: Bard Peripheral Vascular, Inc., Franklin Lakes, NJ (US)

(72) Inventors: Hely Li, Shanghai (CN); Feipeng Guo, Shanghai (CN); Peng Guo, Shanghai (CN)

(21) Appl. No.: 19/053,842

(22) Filed: Feb. 14, 2025

(51) Int. Cl. A61B 8/00; A61B 8/12

(52) U.S. Cl. CPC A61B 8/445; A61B 8/12; A61B 8/54

(57) ABSTRACT

Embodiments disclosed herein are intravascular ultrasound probe assemblies and related systems and methods of manufacture and use. In an embodiment, a transducer assembly for an intravascular ultrasound system includes a housing, a transducer, an impedance backing layer, and a cable. The housing has a proximal end region configured to secure to a distal end region of a catheter shaft and one or more interior walls defining a through hole. The transducer is positioned within the through hole of the housing. The impedance backing layer is positioned within the through hole of the housing. The cable is secured to the transducer and extends through the housing. The cable is configured operably couple to a control system of the intravascular ultrasound system.

### BACKGROUND

Intravascular ultrasound (“IVUS”) is an intravascular imaging modality that can be used in various interventional disciplines to characterize lesion morphology, quantify plaque load, guide device sizing, evaluate device placement, and identify complications. IVUS provides cross-sectional images of vascular structures. IVUS can play an important role in decision making during percutaneous coronary intervention (“PCI”) procedures, including pre-procedure evaluation and post-procedure optimization. IVUS is used to assess plaque nature and characteristics, develop appropriate pretreatment strategies, measure lesion length and reference segment vessel diameter, and select appropriate stent diameter, length, and footing points. Post-PCI, IVUS is used to identify stent expansion and apposition and complications such as stent edge entrapment in order to target and optimize the immediate post-PCI outcomes.

### SUMMARY

Embodiments disclosed herein are intravascular ultrasound probe assemblies, and related systems and methods of assembly. In an embodiment, a transducer assembly for an intravascular ultrasound system includes a housing, a transducer, an impedance backing layer, and a cable. The housing has a proximal end region configured to secure to a distal end region of a catheter shaft. The housing also has one or more interior walls defining a through hole. The transducer is positioned within the through hole of the housing. The impedance backing layer is positioned within the through hole of the housing. The cable is secured to the transducer and extending through the housing, the cable being configured operably couple to a control system of the intravascular ultrasound system.

In an embodiment, an imaging catheter for an intravascular ultrasound system includes a shaft having a distal end region, a housing, a transducer, an impedance backing layer, and a cable. The housing has a proximal end region secured to the distal end region of the shaft, the housing having one or more interior walls defining a through hole. The transducer is positioned within the through hole of the housing. The impedance backing layer is positioned within the through hole of the housing. The cable is secured to the transducer and extends through the housing, the cable being configured operably couple to a control system of the intravascular ultrasound system.

In an embodiment, an intravascular ultrasound system includes a control system and an imaging catheter operably coupled to the control system. The imaging catheter includes a shaft having a distal end region, a housing, a transducer, an impedance backing layer, and a cable. The housing has a proximal end region secured to the distal end region of the shaft, the housing having one or more interior walls defining a through hole. The transducer is positioned within the through hole of the housing. The impedance backing layer is positioned within the through hole of the housing. The cable is secured to the transducer and extends through the housing, the cable operably coupled to the control system.

In an embodiment, a method of assembling a transducer assembly of an intravascular ultrasound system is disclosed. The method includes providing a housing having a proximal end region configured to secure to a distal end region of a catheter shaft, the housing having one or more interior walls defining a through hole. The method also includes positioning a transducer within the through hole of the housing, securing a cable to the transducer, at least partially filling the through hole with a backing layer material to cover an inward facing surface of the transducer in the through hole, and curing the backing layer material to form an impedance backing layer material.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of an IVUS system, according to an embodiment.

FIG. 2 is a side view of a portion of an imaging catheter of the IVUS system of FIG. 1, according to an embodiment.

FIG. 3A is an exploded side view of the probe of the imaging catheter of FIG. 2, according to an embodiment.

FIG. 3B is a top view of the probe of FIG. 2 before the impedance backing layer is secured to the probe, according to an embodiment.

FIG. 3C is a top view of the probe of FIG. 2 with the impedance backing layer secured to the probe, according to an embodiment.

FIG. 4 is a flow diagram of a method of manufacturing a transducer assembly of an intravascular ultrasound system, according to an embodiment.

### DETAILED DESCRIPTION

Embodiments disclosed herein include IVUS probe assemblies of imaging catheters, and related systems and methods. The IVUS probe assemblies disclosed herein include an ultrasonic transducer assembly that is more efficient to assemble than conventional IVUS probe assemblies. The probe of the imaging catheter of the IVUS systems disclosed herein is configured to allow the transducer (e.g., ultrasonic transducer slide) to emit an ultrasonic wave to an interface, such as a blood vessel interface, and receives the signals reflected by the interface. The control system of the IVUS system then may convert the signals from the probe of the imaging catheter to images in a real-time manner.

For high performance, a transducer assembly emits and receives high quality signals. This can require (1) the transducer slide be parallel to the rotation axial of the probe, (2) the back side of the transducer slice should be able to shield ultrasonic signal as much as possible to avoid noise, and (3) there should be a thin layer on front of transducer slice matching the acoustic impedances between transducer and human blood. The probe of the imaging catheter may include a housing, a transducer (e.g., transducer slice), one or more cables, an impedance backing layer, a front matching layer, and a shaft (e.g., coil). The housing configurations and the methods of assembling probes of the imaging catheters disclosed herein result in more effective and reliably consistent probes.

In many embodiments, the housing of the imaging catheter includes a substantially planar side surface on the front and a rectangle or other shaped (e.g., oval) through hole sized to accommodate the transducer (e.g., transducer slice). When assembling the transducer assembly, the substantially planar side surface may be placed on to a working plane, with the planar side surface oriented downward. The transducer may then be placed in the rectangular space of the through hole with the transducer spaced from the walls of the housing defining the through hole such that the transducer does not contact the housing (e.g., the transducer is placed without direct circumferential contact with the housing). This positioning of the transducer in the through hole allows the transducer and the substantially planar side surface on the front of the housing to be substantially (e.g., exactly) coplanar. The coplanar positioning of the transducer and the substantially planar side surface on the front of the housing allows the ultrasonic signal to be emitted and received without shifting of incidence angle, thereby improving the signal strength with a mechanical configuration instead of complex signal processing.

With the transducer being spaced from the housing such that the transducer does not directly contact the housing, electric short circuiting that can result in the probe not working at all will not occur. Moreover, the transducer slide would otherwise be damaged when the transducer vibrates due to the friction with the housing. The through hole and planar side surface configuration of the housing allows for more precise and simpler assembling of transducer assembly and housing.

In many embodiments, after fixing the position of transducer relative to the housing, one can choose either welding the cables onto the transducer or using conductive plastics. With the one or more cables secured to the transducer, at least partially filling the through hole with a fluidic backing layer material is easier and more controllable. As the backing layer material cures to form the impedance backing material, application of the front matching layer is more controllable because the transducer and substantially planar side surface on the front of the housing are substantially coplanar. In many embodiments, the substantially planar side surface on the front of the housing can accommodate a transducer with a larger area. The coplanar positioning of the transducer and the planar side surface of the housing allow for the emitting and receiving of the ultrasonic signal without blockages.

Conventional imaging catheters are typically difficult to assemble. For example, the housing of conventional imaging catheters has a recess rather than a through hole, thus requiring the backing layer to be filled into the recess before the transducer is positioned on the front of the backing layer. In this process, it is difficult to maintain the transducer at a desired position, such as parallel to a longitudinal axis of the probe of the imaging catheter. In conventional imaging catheters, the thickness of the backing layer is not adjustable because of the recess size of the housing. Accordingly, to make a precise backing layer in a conventional imaging catheter, the composition of the backing layer material must be adjusted. Moreover, the configuration of the housing of conventional imaging catheters and transducer assemblies allows for a relatively smaller transducer than the assemblies disclosed herein, resulting in the conventional transducer assembly emitting and receiving fewer effective signals.

In contrast, the imaging catheters and transducer assemblies disclosed herein are easier to assemble, and improve assembly yields in comparison to conventional imaging catheters and transducer assemblies. Moreover, the imaging catheters disclosed herein are able to accommodate a larger transducer than conventional imaging catheters, resulting in more signals obtained by the transducers to generate a larger and clearer image. The methods of assembly the imaging catheters and transducer assemblies disclosed herein allow for a simple filling of a backing layer material, as well as an adjustable thickness of the backing layer. In many embodiments, the coplanar positioning of the substantially planar side surface on the front of the housing and the transducer result in the absence of signal shielding in the imaging catheter.

IVUS systems disclosed herein may be used in various interventional disciplines to characterize lesion morphology, quantify plaque load, guide device sizing, evaluate device placement, and identify complications. IVUS systems disclosed herein are configured to provide cross-sectional images of vascular structures. In many embodiments, IVUS systems disclosed herein may play an important role in decision making during percutaneous coronary intervention (“PCI”) procedures, including pre-procedure evaluation and post-procedure optimization. In many embodiments, IVUS systems disclosed herein are configured for use in peripheral vascular diagnosis. IVUS systems disclosed herein may be used to assess plaque nature and characteristics, develop appropriate pretreatment strategies, measure lesion length and reference segment vessel diameter, and select appropriate stent diameter, length, and footing points. Post-PCI, IVUS systems disclosed herein may be used to identify stent expansion and apposition and complications such as stent edge entrapment in order to target and optimize the immediate post-PCI outcomes.

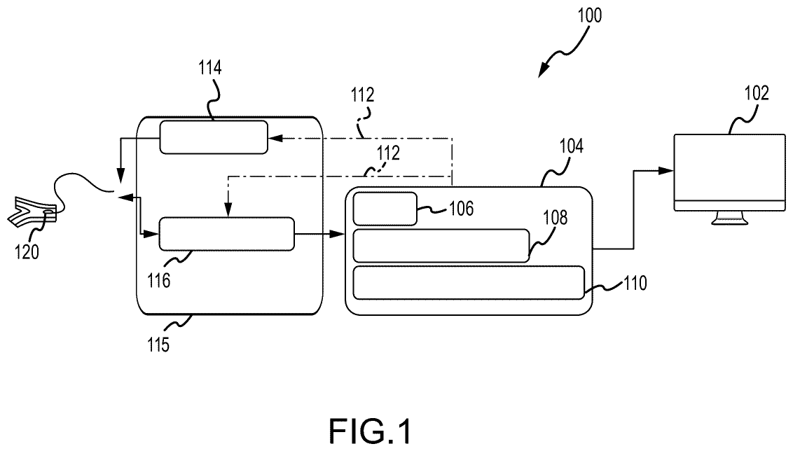

Turning now to the drawings, FIG. 1 is a block diagram of an IVUS system 100, according to an embodiment. The IVUS system may include a control system 104 (e.g., console or integrated power center (“IPC”)), an automatic pullback device 115 operably coupled to the control system 104, and an imaging catheter 120 operably coupled to the pullback device 115. The control system 104 may be part of an electronic device or computer 102 and/or may be operably coupled thereto. The control system 104 may include one or more processors or modules, such as a data acquisition module 106, a signal processing module 108, and an imaging reconstruction module 110. The control system 104 may be electrically or otherwise operably coupled 112 to the automatic pullback device 115. The automatic pullback device may include one or more motors 114 and one or more circuits 116. The one or more circuits may include, for example, a transmitter circuit and/or a receiver circuit operably coupled to the imaging catheter 120 and the control system 104. The pullback device 115 may be configured to pull back the imaging catheter 120 (e.g., the transducer of the imaging catheter 120) along a length of the vessel to allow the imaging catheter 120 to emit and collect ultrasonic signals to form multiple image slices of the vascular cross section that may be integrated to generate a three-dimensional image.

In order to produce the desired image, the imaging catheter 120 may include a transducer at a distal end region thereof that receives an electrical signal from the control system 104, and the electrical stimulation of the electrical signal causes the piezoelectric element in the transducer to expand and contract to produce high-frequency ultrasound waves. These high-frequency ultrasound waves may be scattered and reflected at the tissue interface, and some of the reflected ultrasound waves are converted back to electrical signals by the transducer. These electrical signals from the reflected ultrasound waves may be analyzed and processed by the imaging reconstruction module 110 in the control system 104 for conversion into grayscale cross-sectional images.

The control system 104 may be used to carry out any of the example methods disclosed herein, such as using a controller. The control system 104 may include at least one computing device. The at least one computing device of the control system 104 may be configured to perform one or more of the acts described herein. The at least one computing device of the control system 104 can include one or more servers, one or more computers (e.g., desk-top computer, lap-top computer), or one or more mobile computing devices (e.g., smartphone, tablet, etc.). In some embodiments, the computing device of the control system 104 comprises the computer 102. The computing device of the control system 104 may include one or more (e.g., all) of the data acquisition module 106, the signal processing module 108, and/or the imaging reconstruction module 110. The computing device of the control system 104 can comprise at least one processor, memory, a storage device, an input/output (“I/O”) device/interface, and a communication interface. In some examples, the at least one computing device of the control system 104 may include a plurality of computing devices, such as a server farm, computational network, or cluster of computing devices.

In some examples, the computing device of the control system 104, the data acquisition module 106, the signal processing module 108, and/or the imaging reconstruction module 110 may include processor(s) having hardware for executing instructions (e.g., instructions for carrying out one or more portions of any of the methods disclosed herein), such as those making up a computer program. For example, to execute instructions, the processor(s) may retrieve (or fetch) the instructions from an internal register, an internal cache, the memory, or a storage device and decode and execute them. In particular examples, processor(s) may include one or more internal caches for such data. As an example, the processor(s) may include one or more instruction caches, one or more data caches, and one or more translation lookaside buffers (“TLBs”). Instructions in the instruction caches may be copies of instructions in memory or a storage device. In some examples, the processor may be configured (e.g., include programming stored thereon or executed thereby) to carry out one or more portions of any of the example methods disclosed herein.

In some examples, the processor of the control system 104 is configured to perform any of the acts disclosed herein or cause one or more portions of the computing device or control system 104 to perform at least one of the acts disclosed herein. Such configuration can include one or more operational programs (e.g., computer program products) that are executable by the at least one processor.

The at least one computing device (e.g., a server) of the control system 104 may include at least one memory storage medium (e.g., memory and/or storage device). The computing device of the control system 104 may include memory, which is operably coupled to the processor(s). The memory may be used for storing data, metadata, and programs for execution by the processor(s). The memory may include one or more of volatile and non-volatile memories, such as Random Access Memory (“RAM”), Read Only Memory (“ROM”), a solid state disk (“SSD”), Flash, Phase Change Memory (“PCM”), or other types of data storage. The memory may be internal or distributed memory.

The computing device of the control system 104 may include the storage device having storage for storing data or instructions. The storage device may be operably coupled to the at least one processor. In some examples, the storage device can comprise a non-transitory memory storage medium, such as any of those described above. The storage device (e.g., non-transitory storage medium) may include a hard disk drive (“HDD”), a floppy disk drive, flash memory, an optical disc, a magneto-optical disc, magnetic tape, or a Universal Serial Bus (“USB”) drive or a combination of two or more of these. The storage device may include removable or non-removable (or fixed) media. The storage device may be internal or external to the computing device. In some examples, the storage device may include non-volatile, solid-state memory. In some examples, the storage device may include read-only memory (“ROM”). Where appropriate, this ROM may be mask programmed ROM, programmable ROM (“PROM”), erasable PROM (“EPROM”), electrically erasable PROM (“EEPROM”), electrically alterable ROM (“EAROM”), or flash memory or a combination of two or more of these. In some examples, one or more portions of the memory and/or storage device (e.g., memory storage medium(s)) may store one or more databases thereon.

In some examples of the control system 104, data may be stored in a memory storage medium such as one or more of the at least one processor (e.g., internal cache of the processor), memory, or the storage device. In some examples, the at least one processor may be configured to access (e.g., via a bus) the memory storage medium(s) such as one or more of the memory or the storage device. For example, the at least one processor may receive and store the data (e.g., look-up tables) as a plurality of data points in the memory storage medium(s). The at least one processor may execute programming stored therein adapted access the data in the memory storage medium(s). For example, the at least one processor may access data in the memory storage medium(s) such as memory or storage device.

The computing device of the control system 104 also may include one or more I/O devices/interfaces, which are provided to allow a user to provide input to, receive output from, and otherwise transfer data to and from the computing device. These I/O devices/interfaces may include a mouse, keypad or a keyboard, a touch screen, camera, optical scanner, network interface, web-based access, modem, a port, other known I/O devices, or a combination of such I/O devices/interfaces. The touch screen may be activated with a stylus or a finger. The I/O devices/interfaces may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces are configured to provide graphical data to a display for presentation to a user. The graphical data may be representative of one or more graphical user interfaces and/or any other graphical content as may serve a particular implementation.

The computing device of the control system 104 can further include a communication interface. The communication interface can include hardware, software, or both. The communication interface can provide one or more interfaces for communication (such as, for example, packet-based communication) between the computing device and one or more additional computing devices or one or more networks. For example, the communication interface may include a network interface controller (“NIC”) or network adapter for communicating with an Ethernet or other wire-based network or a wireless NIC (“WNIC”) or wireless adapter for communicating with a wireless network, such as a WI-FI.

Any suitable network and any suitable communication interface may be used. For example, the computing device of the control system 104 may communicate with an ad hoc network, a personal area network (“PAN”), a local area network (“LAN”), a wide area network (“WAN”), a metropolitan area network (“MAN”), or one or more portions of the Internet or a combination of two or more of these. One or more portions of one or more of these networks may be wired or wireless. As an example, one or more portions of the control system 104 or the computing device of the control system 104 may communicate with a wireless PAN (“WPAN”) (such as, for example, a BLUETOOTH WPAN), a WI-FI network, a WI-MAX network, a cellular telephone network (such as, for example, a Global System for Mobile Communications (“GSM”) network), or other suitable wireless network or a combination thereof. The computing device of the control system 104 may include any suitable communication interface for any of these networks, where appropriate.

The computing device of the control system 104 may include a bus. The bus can include hardware, software, or both that couples components of the computing device of the control system 104 to each other. For example, the bus of the control system 104 may include an Accelerated Graphics Port (“AGP”) or other graphics bus, an Enhanced Industry Standard Architecture (“EISA”) bus, a front-side bus (“FSB”), a HYPERTRANSPORT (“HT”) interconnect, an Industry Standard Architecture (“ISA”) bus, an INFINIBAND interconnect, a low-pin-count (“LPC”) bus, a memory bus, a Micro Channel Architecture (“MCA”) bus, a Peripheral Component Interconnect (“PCI”) bus, a PCI-Express (“PCIe”) bus, a serial advanced technology attachment (“SATA”) bus, a Video Electronics Standards Association local (“VLB”) bus, or another suitable bus or a combination thereof.

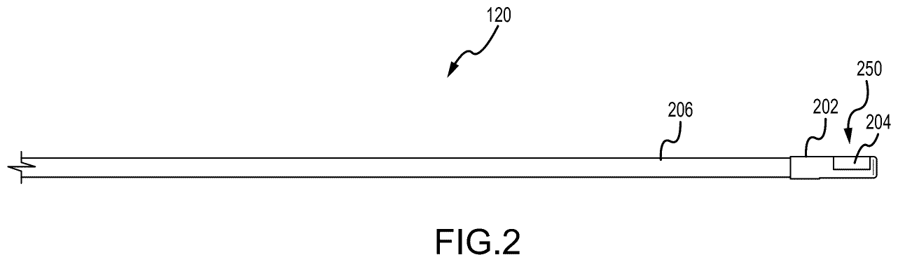

Turning ahead in the drawings, FIG. 2 is a side view of a portion of an imaging catheter 120 of the IVUS system 100 of FIG. 1, according to an embodiment. The imaging catheter 120 may include a shaft 206 (such as a torque coil), a housing 202, and a probe comprising a transducer assembly 250 having a matching layer 204. The imaging catheter 120 may be sized and dimensioned to be inserted into and fit within a vessel. In operation with the system 100 the imaging catheter 120 may be used in percutaneous coronary intervention (“PCI”) procedures, including pre-procedure evaluation and post-procedure optimization. The imaging catheter 120 of the IVUS system 100 may be inserted into a vessel and used to assess plaque nature and characteristics, develop appropriate pretreatment strategies, measure lesion length and reference segment vessel diameter, and select appropriate stent diameter, length, and footing points. Post-PCI, the imaging catheter 120 may be used to identify stent expansion and apposition and complications such as stent edge entrapment in order to target and optimize the immediate post-PCI outcomes.

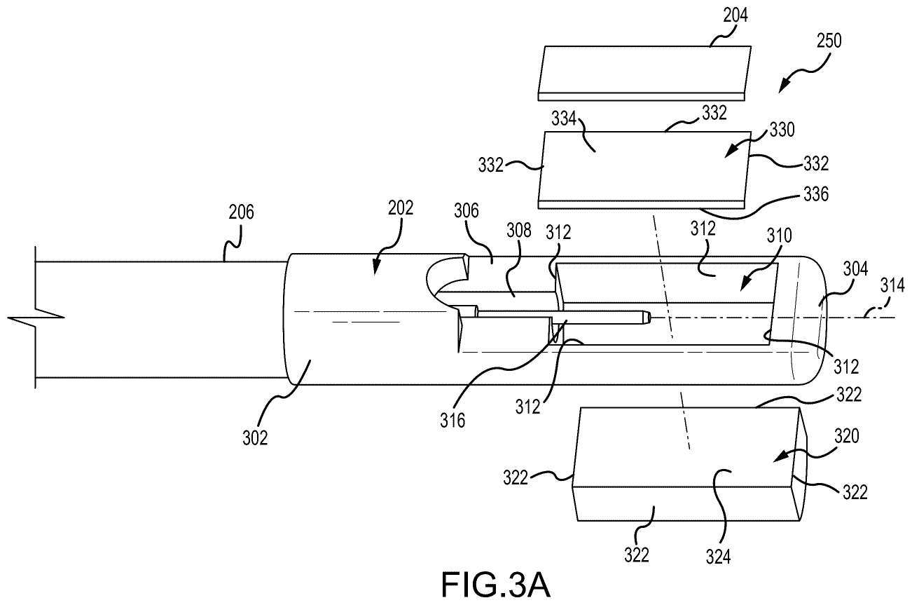

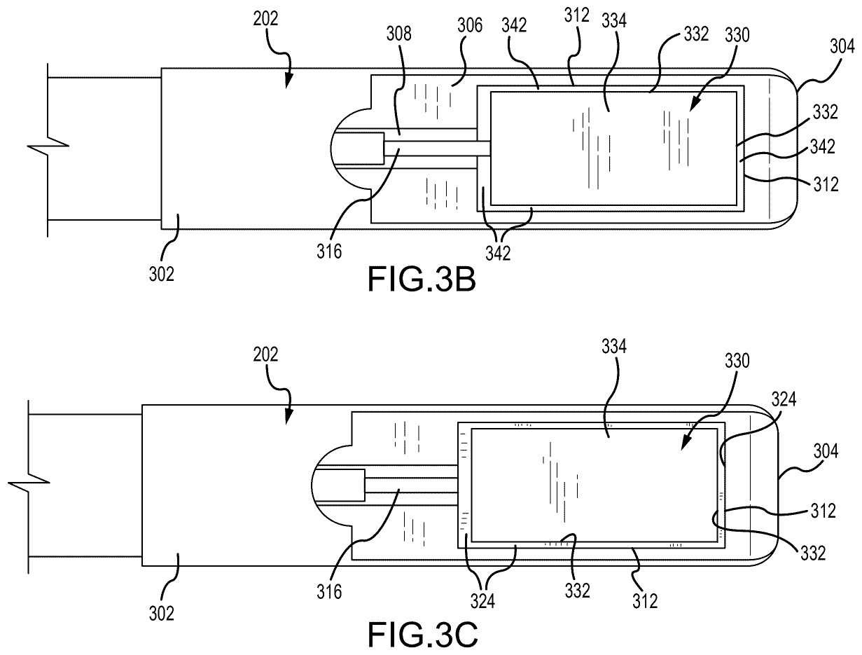

Turning ahead in the drawings, FIG. 3A is an exploded side view of the transducer assembly 250 of the probe of the imaging catheter 120, FIG. 3B is a top view of the transducer assembly 250 of the probe of the imaging catheter 120 before an impedance backing layer 320 is secured to the transducer assembly 250, and FIG. 3C is a top view of the transducer assembly 250 of the probe of the imaging catheter 120 with the impedance backing layer 320 secured to the transducer assembly 250, according to an embodiment. The transducer assembly 250 may include the housing 202, a transducer 303, an impedance backing layer 320, the matching layer 204, and a cable 316 electrically coupled or coupleable to the control system 104.

The housing 202 has a proximal end region 302 configured to secure to a distal end region of a shaft 206 (e.g., catheter shaft), a distal end region 304, and one or more interior walls 312 defining a through hole 310 extending through the housing 202. In some embodiments, the one or more interior walls 312 may include four interior walls 312 defining a substantially rectangular through hole 310 or other shaped through hole (e.g., elliptical). In other embodiments, the housing 202 may include any number of interior walls 312 defining through holes of other shapes and configurations. The housing 202 also may include a substantially planar side surface 306 extending in a longitudinal direction at least partially between the distal end region 304 and the proximal end region 302. In some embodiments, the side surface 306 extends proximally from the distal end region 304. The side surface 306 is substantially parallel to the longitudinal axis 314 of the housing 202 and/or the imaging catheter 120. The through hole 310 may extend through the substantially planar side surface 306 to a surface of the housing 202 that is substantially opposite to the side surface 306. The housing 202 also may include a slot 308 or channel extending proximally from the through hole 310 at least partially to the shaft 206. In some embodiments, the slot 308 may be aligned substantially along a longitudinal axis 314 of the imaging catheter 120. The slot 308 also may be recessed from the side surface 308. In some embodiments, the housing 202 may include plastic or stainless steel.

The transducer assembly 250 also includes a transducer 330 positioned within the through hole 310 of the housing 202. The transducer 330 may include a piezoelectric element(e.g., ultrasonic transducer slide), such as a lead magnesium niobate-lead titanate (“PMN-PT”) piezoelectric element, inorganic piezoelectric elements (e.g., lead zirconate titanate, zinc oxide, and/or hexagonal boron nitride flakes), organic piezoelectric elements (e.g., polyvinylidene fluoride), composite piezoelectric elements including inorganic (e.g., lead zirconate titanate, zinc oxide, and/or hexagonal boron nitride flakes) and organic materials (e.g., polyvinylidene fluoride), bio-inspired materials, or combinations thereof. The transducer 330 includes an outward oriented surface 334, an inward oriented surface 336, and one or more peripheral edges 332.

In some embodiments, the transducer 330 may include a shape that is generally complementary to the shape of the through hole 310, but smaller. For example, the one or more peripheral edges 332 that are substantially parallel to the one or more interior walls 312 of the housing 202 defining the through hole 310. In some embodiments, the transducer 330 may be substantially rectangular and include four peripheral edges 332 substantially parallel with the four interior walls 312 of the housing 202. With the transducer 330 being sized smaller than the through hole 310, the one or more peripheral edges 332 may be spaced from the one or more interior walls 312 of the housing 202 defining the through hole 310. Accordingly, the transducer 330 does not contact the one or more interior walls 312 of the housing 202 defining the through hole 310, according to one or more embodiments. As shall be described in greater detail below and as illustrated in FIG. 3B, a gap or space 342 may be present between the one or more peripheral edges 332 and the one or more interior walls 312 of the housing 202 before the impedance backing layer 320 is secured to the transducer assembly 250. In some embodiments, at least the outward oriented surface 334 is substantially planar. In embodiments having a substantially planar side surface 306, the outward oriented surface 334 of the transducer 330 may be substantially coplanar with the substantially planar side surface 306 of the housing 202. The inward oriented surface 336 also may be substantially planar.

The matching layer 204 may cover the transducer 330. In some embodiments, the matching layer 204 may be positioned on top of the substantially planar side surface 306 and the outward oriented surface 334 of the transducer 330, with the matching layer 204 being substantially parallel to the substantially planar side surface 306 and the outward oriented surface 334 of the transducer 330. The matching layer 204 may include an acoustic matching layer 204. The matching layer 204 is configured to help transfer ultrasound energy and may include one or more materials conducive to achieve energy transfer, such as epoxy, polyurethane, polystyrene, or combinations thereof.

The transducer assembly 250 also includes the impedance backing layer 320 positioned within the through hole 310 of the housing 202. In many embodiments, the impedance backing layer 320 covers the inward oriented surface 336 of the transducer 330. For example, the impedance backing layer 320 may include an inward oriented surface 324 that interfaces or is adjacent to the inward oriented surface 336 of the transducer 330. The impedance backing layer 320 also may fill in the gap or space 342 between the one or more peripheral edges 332 and the one or more interior walls 312 defining the through hole 310 such that the impedance backing layer is at least partially positioned between the one or more peripheral edges 332 of the transducer 330 and the one or more interior walls 312 of the housing 202. The impedance backing layer may secure the transducer 330 to the housing 202. The periphery 322 of the impedance backing layer 320 may be adjacent to or interfacing the interior walls 312.

The impedance backing layer 320 may include a conductive glue or adhesive. For example, the impedance backing layer 320 may include a glue, adhesive, or other material that may be cured to secure the transducer 330 to within the through hole 310 of the housing 202. In some embodiments, the impedance backing layer 320 may include epoxy with tungsten powder and/or silver in the backing layer 320 to absorb the signals from the non-working side of the transducer assembly 250.

The imaging catheter 120 also may include the cable 316 secured to the transducer 330 and extending through the housing 202. In some embodiments, the cable 316 is positioned within the slot 308 or channel in the housing 202. The cable 316 is operably coupled or configured to be operably coupled to the control system 104 of the IVUS system 100. In order to produce the desired image from the imaging catheter 120, the transducer 330 may receive one or more electrical signals from the control system 104, and the electrical stimulation of the one or more electrical signals causes the piezoelectric element in the transducer 330 to expand and contract to produce high-frequency ultrasound waves. These high-frequency ultrasound waves may be scattered and reflected at the tissue interface, and some of the reflected ultrasound waves are converted back to electrical signals by the transducer 330. These electrical signals from the reflected ultrasound waves may be analyzed and processed by the imaging reconstruction module 110 in the control system 104 for conversion into grayscale cross-sectional images.

Also disclosed herein are imaging catheters and IVUS systems that include the any embodiments of the transducer assemblies and probes described above. For example, the imaging catheter 120 (shown in FIG. 2) may include a shaft 206 having a distal end region, the housing 202 secured to the distal end region of the shaft 206, the cable 304 (shown in FIGS. 3A-3C) extending through the shaft 120 and operably coupled or configured to operably couple to the control system 104 (shown in FIG. 1) of the IVUS system 100, and a probe including the transducer assembly 250 secured to the housing 202.

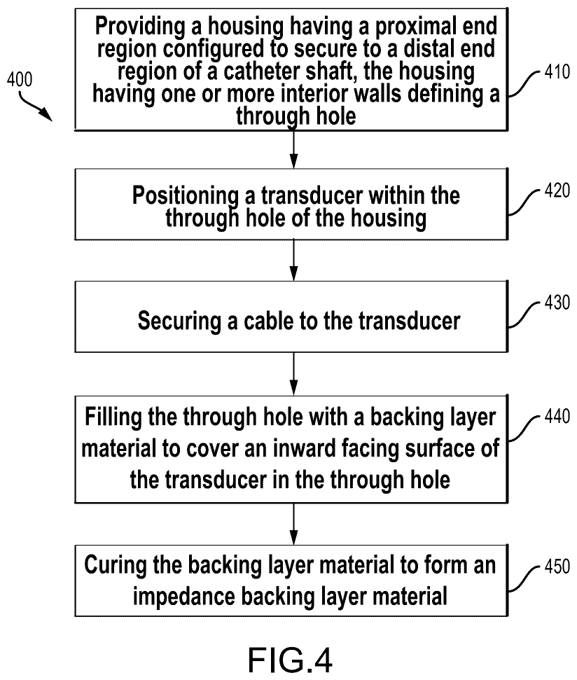

Turning ahead in the drawings, FIG. 4 is a flow diagram of a method 400 of assembling or manufacturing a transducer assembly of an IVUS system, according to an embodiment. The transducer assembly assembled according to the method 400 may include any of the transducer assemblies of the probes disclosed herein. In many embodiments, the method 400 includes providing 410 a housing having a proximal end region configured to secure to a distal end region of a catheter shaft, the housing having one or more interior walls defining a through hole. The method 400 also may include positioning 420 a transducer within the through hole of the housing, securing 430 a cable to the transducer, at least partially filling 440 the through hole with a backing layer material to cover an inward facing surface of the transducer in the through hole, and curing 450 the backing layer material to form an impedance backing layer material. Acts of the method 400 are for illustrative purposes. For example, acts of the method 400 may be performed in different orders, split into multiple acts, modified, supplemented, or combined.

In many embodiments of the method 400, positioning 420 a transducer within the through hole of the housing includes positioning the transducer within the through hole of the housing such that the transducer does not contact the one or more interior walls of the housing defining the through hole. For example, in many embodiments, the housing of the imaging catheter includes a substantially planar side surface on the front and a rectangle through hole sized to accommodate the transducer. Positioning 420 the transducer within the through hole of the housing may include positioning the transducer in the rectangular space of the through hole with the transducer spaced from the walls of the housing defining the through hole such that the transducer does not contact the housing (e.g., the transducer is placed without direct circumferential contact with the housing). This positioning of the transducer in the through hole allows the transducer and the substantially planar side surface on the front of the housing to be substantially (e.g., exactly) coplanar. Accordingly, positioning 420 a transducer within the through hole of the housing may include positioning the transducer within the through hole of the housing with an outward oriented surface of the transducer being substantially coplanar with a substantially planar region of the housing having the through hole extending therethrough.

In the finished probe of the imaging catheter including the transducer assembly of the method 400, the coplanar positioning of the transducer and the substantially planar side surface on the front of the housing allows the ultrasonic signal to be emitted and received without shifting of incidence angle, thereby improving the signal strength with a mechanical configuration instead of complex signal processing. With the transducer being spaced from the housing such that the transducer does not directly contact the housing, electric short circuiting that results in the probe not working at all will not occur. Moreover, the transducer slide would otherwise be damaged when the transducer vibrates due to the friction with the housing. The through hole and planar side surface configuration of the housing allows for more precise and simpler assembling of transducer assembly and housing.

The method 400 also may include securing a cable to the transducer. In many embodiments, after fixing the position of transducer relative to the housing, one or more cables may either be welded onto the transducer or secured to the transducer with one or more conductive plastics. In some embodiments, before securing the cable and filling in the backing layer material, the transducer is placed on an adhesive plate. The housing may then be placed on top of the transducer, with the housing adjusted such that the transducer is positioned within the through hole without touching the housing.

With the one or more cables secured to the transducer, at least partially filling 440 the through hole with a backing layer material to cover an inward facing surface of the transducer in the through hole is easier and more controllable. In many embodiments of the method 400, at least partially filling 440 the through hole with a backing layer material to cover an inward facing surface of the transducer in the through hole includes filling the through hole with the backing layer material to cover the inward facing surface of the transducer in the through hole and position the backing layer material between one or more peripheral edge of the transducer and the one or more interior walls of the housing.

In many embodiments, the method 400 further includes securing a matching layer to the housing to cover the transducer. As the backing material cures to form the impedance backing layer, application of the front matching layer is more controllable because the transducer and substantially planar side surface on the front of the housing are substantially coplanar.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

1. A transducer assembly for an intravascular ultrasound system, the transducer assembly comprising:
a housing having a proximal end region configured to secure to a distal end region of a catheter shaft, the housing having a first side, a second side opposite to the first side, and one or more interior walls defining a through hole extending through the housing from the first side to the second side;
a transducer positioned within the through hole of the housing at least partially between the first side and the second side of the housing;
an impedance backing layer positioned within the through hole of the housing at least partially between the first side and the second side of the housing; and
a cable secured to the transducer and extending through the housing, the cable being configured operably couple to a control system of the intravascular ultrasound system.

2. The transducer assembly of claim 1, wherein the transducer does not contact the one or more interior walls of the housing defining the through hole.

3. The transducer assembly of claim 2, wherein:
the transducer includes an outward oriented surface, an inward oriented surface, and one or more peripheral edges spaced from the one or more interior walls of the housing defining the through hole; and
the impedance backing layer covers the inward oriented surface of the transducer and is positioned between the one or more peripheral edges of the transducer and the one or more interior walls of the housing.

4. The transducer assembly of claim 3, wherein the first side of the housing includes a substantially planar region having the through hole extending therethrough, the outward oriented surface of the transducer being substantially coplanar with the substantially planar region of the housing.

5. The transducer assembly of claim 1, further comprising a matching layer covering the transducer.

6. An imaging catheter for an intravascular ultrasound system, the imaging catheter comprising:
a shaft having a distal end region;
a housing having a proximal end region secured to the distal end region of the shaft, the housing having a first side, a second side opposite to the first side, and one or more interior walls defining a through hole extending through the housing from the first side to the second side;
a transducer positioned within the through hole of the housing at least partially between the first side and the second side of the housing;
an impedance backing layer positioned within the through hole of the housing at least partially between the first side and the second side of the housing; and
a cable secured to the transducer and extending through the housing, the cable being configured operably couple to a control system of the intravascular ultrasound system.

7. The imaging catheter of claim 6, wherein the transducer does not contact the one or more interior walls of the housing defining the through hole.

8. The imaging catheter of claim 7, wherein:
the transducer includes an outward oriented surface, an inward oriented surface, and one or more peripheral edges spaced from the one or more interior walls of the housing defining the through hole; and
the impedance backing layer covers the inward oriented surface of the transducer and is positioned between the one or more peripheral edges of the transducer and the one or more interior walls of the housing.

9. The imaging catheter of claim 8, wherein the first side of the housing includes a substantially planar region having the through hole extending therethrough, the outward oriented surface of the transducer being substantially coplanar with the substantially planar region of the housing.

10. The imaging catheter of claim 6, further comprising a matching layer covering the transducer.

11. An intravascular ultrasound system, comprising:
a control system; and
an imaging catheter operably coupled to the control system, the imaging catheter comprising:
a shaft having a distal end region;
a housing having a first side, a second side opposite to the first side, and a proximal end region secured to the distal end region of the shaft, the housing having one or more interior walls defining a through hole extending through the housing from the first side to the second side;
a transducer positioned within the through hole of the housing at least partially between the first side and the second side of the housing;
an impedance backing layer positioned within the through hole of the housing at least partially between the first side and the second side of the housing; and
a cable secured to the transducer and extending through the housing, the cable operably coupled to the control system.

12. The intravascular ultrasound system of claim 11, wherein the transducer does not contact the one or more interior walls of the housing defining the through hole.

13. The intravascular ultrasound system of claim 12, wherein:
the transducer includes an outward oriented surface, an inward oriented surface, and one or more peripheral edges spaced from the one or more interior walls of the housing defining the through hole; and
the impedance backing layer covers the inward oriented surface of the transducer and is positioned between the one or more peripheral edges of the transducer and the one or more interior walls of the housing.

14. The intravascular ultrasound system of claim 13, wherein the first side of the housing includes a substantially planar region having the through hole extending therethrough, the outward oriented surface of the transducer being substantially coplanar with the substantially planar region of the housing.

15. The intravascular ultrasound system of claim 11, further comprising a matching layer covering the transducer.

16. A method of assembling a transducer assembly of an intravascular ultrasound system, the method comprising:
providing a housing having a proximal end region configured to secure to a distal end region of a catheter shaft, the housing having a first side, a second side opposite to the first side, and one or more interior walls defining a through hole extending through the housing from the first side to the second side;
positioning a transducer within the through hole of the housing at least partially between the first side and the second side of the housing;
securing a cable to the transducer;
at least partially filling the through hole with a backing layer material to cover an inward facing surface of the transducer in the through hole; and
curing the backing layer material to form an impedance backing layer material.

17. The method of claim 16, wherein positioning a transducer within the through hole of the housing includes positioning the transducer within the through hole of the housing such that the transducer does not contact the one or more interior walls of the housing defining the through hole.

18. The method of claim 17, wherein at least partially filling the through hole with a backing layer material to cover an inward facing surface of the transducer in the through hole includes at least partially filling the through hole with the backing layer material to cover the inward facing surface of the transducer in the through hole and position the backing layer material between one or more peripheral edges of the transducer and the one or more interior walls of the housing.

19. The method of claim 16, wherein positioning a transducer within the through hole of the housing includes positioning the transducer within the through hole of the housing with an outward oriented surface of the transducer being substantially coplanar with a substantially planar region of the first side of the housing having the through hole extending therethrough.

20. The method of claim 16, further comprising securing a matching layer to the housing to cover the transducer.
